(19) United States
(12) Patent Application Publication
(10) Pub. No.: US 2018/0199866 A1
(43) Pub. Date: Jul. 19, 2018

(54) DEVICES WITH REDUCED WICKING VOLUME BETWEEN SENSORS AND SWEAT GLANDS

(71) Applicant: Eccrine Systems, Inc., Cincinnati, OH (US)

(72) Inventor: Jason HEIKENFELD, Cincinnati, OH (US)

(73) Assignee: Eccrine Systems, Inc., Cincinnati, OH (US)

(21) Appl. No.: 15/746,452

(22) PCT Filed: Jul. 23, 2016
(86) PCT No.: PCT/US2016/043771

§ 371 Date: Jan. 22, 2018

Related U.S. Application Data
(60) Provisional application No. 62/196,541, filed on Jul. 24, 2015.
(60) Provisional application No. 62/208,171, filed on Aug. 21, 2015.

(51) Int. Cl. A61B 5/145; A61B 5/1477

(52) U.S. Cl. CPC A61B 5/14517; A61B 5/0531; A61B 5/1477; A61B 5/14521

(57) ABSTRACT

The disclosed invention provides a sweat sensing device configured with reduced volume between sweat sensors and sweat glands, which decreases the chronologically assured sampling interval. In one embodiment, a sweat sensing device placed on the skin for measuring a property of a sweat analyte includes one or more sweat sensors and a volume-reducing component. The volume-reducing component provides a volume-reduced pathway for sweat between the one or more sweat sensors and sweat glands when the device is positioned on the skin. The volume-reducing component may include a wicking material or other component that at least partially creates the volume-reduced pathway.

### CROSS-REFERENCE TO RELATED APPLICATIONS

This application claims benefit of PCT/US16/43771, filed Jul. 23, 2016, as well as to U. S. Provisional Application No. 62/196,541, filed Jul. 24, 2015 and U. S. Provisional Application No. 62/208,171, filed Aug. 21, 2015, the disclosures of which are hereby incorporated by reference herein in their entirety.

### BACKGROUND OF THE INVENTION

Sweat sensing technologies have enormous potential for applications ranging from athletics, to neonatology, to pharmacological monitoring, to personal digital health, to name a few applications. Sweat contains many of the same biomarkers, chemicals, or solutes that are carried in blood and can provide significant information enabling one to diagnose illness, health status, exposure to toxins, performance, and other physiological attributes even in advance of any physical sign. Furthermore, sweat itself, the action of sweating, and other parameters, attributes, solutes, or features on, near, or beneath the skin can be measured to further reveal physiological information.

If sweat has such significant potential as a sensing paradigm, then why has it not emerged beyond decades-old usage in infant chloride assays for Cystic Fibrosis or in illicit drug monitoring patches? For several years, the majority of medical literature has utilized the slow and inconvenient process of sweat stimulation, collection of a sample, transport of the sample to a lab, and then analysis of the sample by a bench-top machine and a trained expert. This process is so labor intensive, complicated, and costly that in most cases a blood test would be a superior modality, since it is the gold standard for most forms of high performance biomarker sensing. Hence, sweat sensing has not emerged into its fullest opportunity and capability for biosensing, especially for continuous or repeated biosensing or monitoring. Furthermore, attempts at using sweat to sense “holy grails” such as glucose have not yet succeeded to produce viable commercial products, reducing the publicly perceived capability and opportunity space for sweat sensing.

Of all the other physiological fluids used for bio monitoring (e.g. blood, urine, saliva, tears), sweat has arguably the least predictable sampling rate in the absence of technology. However, with proper application of technology, sweat can be made to outperform other non-invasive or less invasive biofluids in predictable sampling. For example, it is difficult to control saliva or tear rate without negative consequences for the user (e.g., dry eyes, tears, dry mouth, or excessive saliva while talking). Urine is also a difficult fluid for physiological monitoring, because it is inconvenient to take multiple urine samples, it is not always possible to take a urine sample when needed, and control of biomarker dilution in urine imposes further significant inconveniences on the user or test subject.

However, the state of art in sweat bio monitoring is in need of additional devices and methods to properly reduce the volume between sensors and skin. Reducing sweat volume is critical for fast sampling times or for sampling during intervals with very low sweat rates. In addition, it also may be critical for prolonged stimulation (i.e., in order to minimize stimulation), and for improving biomarker measurements where a low sweat rate is required to ensure correlation between biomarker concentrations in sweat and those in blood.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sweat sensing technology into intimate proximity with sweat as it is generated. With such an invention, sweat sensing could become a compelling new paradigm as a biosensing platform.

### SUMMARY OF THE INVENTION

The disclosed invention provides a sweat sensing device configured with reduced volume between sweat sensors and sweat glands, which decreases the chronologically assured sampling interval. In one embodiment, a sweat sensing device placed on the skin for measuring a property of a sweat analyte includes one or more sweat sensors and a volume-reducing component. The volume-reducing component provides a volume-reduced pathway for sweat between the one or more sweat sensors and sweat glands when the device is positioned on the skin. The volume-reducing component may include a wicking material or other component that at least partially creates the volume-reduced pathway.

### BRIEF DESCRIPTION OF THE DRAWINGS

The disclosed invention will be further appreciated in light of the following descriptions and drawings in which:

FIG. 1 is a cross-sectional view of at least a portion of a wearable device for sweat biosensing.

FIG. 2 is a cross-sectional view of at least a portion of a wearable device for sweat biosensing.

FIG. 3 is a cross-sectional view of at least a portion of a wearable device for sweat biosensing.

FIG. 4 is a cross-sectional view of at least a portion of a wearable device for sweat biosensing.

FIGS. 5A, 5B, 5C and 5D are cross-sectional views of at least a portion of a device for sweat biosensing with a reduced wicking volume.

FIGS. 6A, 6B and 6C are a cross-sectional view, a partial top view, and a partial cross-sectional view, respectively, of at least a portion of a device for sweat biosensing with a reduced wicking volume.

FIGS. 7A and 7B are a partial top view and a partial cross-sectional view, respectively, of at least a portion of a device for sweat biosensing with a reduced wicking volume.

FIG. 8 is a partial cross-sectional view of at least a portion of a sweat sensing device that further has protection for the surface of a sensor.

FIG. 9 is a partial cross-sectional view of at least a portion of a sweat sensing device that further has protection for the surface of a sensor.

FIG. 10 is a partial cross-sectional view of at least a portion of a sweat sensing device that further has protection for the surface of a sensor.

FIGS. 11A, 11B and 11C are partial cross-sectional views of at least a portion of a sweat sensing device with a reduced wicking volume.

### DEFINITIONS

“Chronological assurance” means a sampling rate or sampling interval for measurement(s) of sweat, or solutes in sweat, at which measurements can be made of new sweat or its new solutes as they originate from the body. Chronological assurance may also include a determination of the effect of sensor function, or potential contamination with previously generated sweat, previously generated solutes, other fluid, or other measurement contamination sources for the measurement(s).

“Sweat sampling rate” means the effective rate at which new sweat, or sweat solutes, originating from the sweat gland or from skin or tissue, reaches a sensor that measures a property of sweat or its solutes. Sweat sampling rate, in some cases, can be far more complex than just sweat generation rate. Sweat sampling rate directly determines, or is a contributing factor in determining chronological assurance. Times and rates are inversely proportional (rates having at least partial units of 1/seconds), therefore a short or small time required to refill a sweat volume can also be said to have a fast or high sweat sampling rate. The inverse of sweat sampling rate (1/s) could also be interpreted as a “sweat sampling interval”. Sweat sampling rates or intervals are not necessarily regular, discrete, periodic, discontinuous, or subject to other limitations. Like chronological assurance, sweat sampling rate may also include a determination of the effect of potential contamination with previously generated sweat, previously generated solutes, other fluid, or other measurement contamination sources for the measurement(s). Sweat sampling rate can also be in whole or in part determined from solute generation, transport, advective transport of fluid, diffusion transport of solutes, or other factors that will impact the rate at which new sweat or sweat solutes reach a sensor and/or are altered by older sweat or solutes or other contamination sources. Sensor response times may also affect sampling rate.

“Sweat generation rate” means the rate at which sweat is generated by the sweat glands themselves. Sweat generation rate is typically measured by the flow rate from each gland in nL/min/gland. In some cases, the measurement is then multiplied by the number of sweat glands from which the sweat is being sampled.

“Measured” may mean an exact or precise quantitative measurement and can include broader meanings such as, for example, measuring a relative amount of change of something. Measured can also mean a binary measurement, such as ‘yes’ or ‘no’ type measurements.

“Sweat volume” means the fluidic volume in a space that can be defined multiple ways. Sweat volume may be the volume that exists between a sensor and the point of generation of sweat, or between a sensor and a solute moving into or out of sweat from the body or from other sources. Sweat volume can include the volume that can be occupied by sweat between the sampling site on the skin and a sensor on the skin, where the sensor has no intervening layers, materials, or components between it and the skin; or between the sampling site on the skin and a sensor on the skin where there are one or more layers, materials, or components between the sensor and the sampling site on the skin. Sweat volume may refer to the sweat volume of multiple integrated components, or used in description of the sweat volume for single component or a subcomponent, or in the space between a device, or device component, and skin.

“Volume-reducing component” means any component, material, element, or feature of the present disclosure that facilitates the creation of a volume-reduced pathway.

“Volume-reduced pathway” means a sweat volume that has been reduced by the addition of a material, device, layer, or other component, which therefore decreases the sweat sampling interval for a given sweat generation rate. Specific to the instant disclosure, a volume reduced pathway refers to any combination of elements disclosed herein that at least in part uses wicking pressure to enable the formation of the volume reduced pathway. For example, a volume reduced pathway could be created in the space between a sweat collector and skin by wicking sweat through this space. The disclosed invention may benefit from additional methods to reduce the sweat volume, but if the term volume-reduced pathway is used herein, then wicking pressure must, at least in part, enable or create the volume-reduced pathway.

“Microfluidic components” means channels in polymer, textiles, paper, or other components known in the art of microfluidics for guiding movement of a fluid or at least partial containment of a fluid.

“Wicking pressure,” “wicking force,” “capillary pressure,” or “capillary force,” means a pressure or force that should be interpreted according to its general scientific meaning. For example, a capillary (tube) geometry can be said to have a capillary pressure or a wicking pressure. Or a wicking textile or gel may have a capillary pressure, even if the material is not geometrically a tube or a channel. Conversely, a wicking fiber can have an effective capillary pressure. Similarly, the (relatively empty) space between a material placed on skin and the skin surface can have an effective wicking pressure. The terms wicking or capillary pressure and wicking or capillary force may be used interchangeably herein to describe the effective pressure provided by any component or material that is capable of capturing sweat by a negative pressure (i.e., pulling it into or along said component or material). For simplicity, the term “wicking pressure” will be used herein to refer to any of the above alternate terms. Wicking pressure also must be considered in its specific context, for example, if a sponge is fully saturated with water, then it has no remaining wicking pressure. Wicking pressure must therefore be interpreted as described in the specification for a device during use, and not interpreted in isolation or in contexts other than the disclosed devices or use scenarios.

“Wicking collector” means any component of the disclosed invention that supports the creation of, or sustains, a volume reduced pathway, or that is the wicking element that receives sweat before a sweat sensing device sensor and is on or adjacent to skin. A wicking collector can be a microfluidic component, a capillary material, a wrinkled surface, a textile, a gel, a coating, a film, or any other component that satisfies the general criteria of the present disclosure. A wicking collector may be part of the same component or material that serves other purposes (e.g., a wicking pump or a wicking coupler), and in such cases, the portion of said component or material that at least in part receives sweat before the sensor(s) and is on or adjacent to skin is also a wicking collector as defined herein.

“Wicking pump” refers to any component of the disclosed invention that supports creation of or sustains a volume reduced pathway, or that receives sweat after a sweat sensing device sensor and has a primary purpose of collecting excess sweat to allow sustained operation of the device. A wicking pump may also include an evaporative material or surface that is configured to remove excess sweat by evaporation of water. A wicking pump may be part of the same component or material that serves other purposes (e.g., a wicking collector or a wicking coupler), and in such cases, the portion of said component or material that at least in part receives sweat after the sensor(s), is also a wicking pump as defined herein.

The term “wicking pump” may also reference alternate configurations, such as a small mechanical pump, or osmotic pressure across a membrane, so long as the pressure generated satisfies the requirements described herein, and the other materials or components between the wicking pump and skin operate by wicking pressure to maintain their respective sweat volumes. For example, a suctioning system that is air-tight to skin would not be considered a wicking pump because the disclosed invention permits introduction of air or gas between the device and skin.

“Wicking coupler” refers to any component of the disclosed invention that is on or adjacent to a sweat sensing device sensor and that promotes coupling and transport of sweat or its solutes by advective flow, diffusion, or other method of transport, between another wicking component or material and at least one device sensor. In some embodiments, the wicking coupler function may be performed by a suitably configured wicking collector. In other embodiments, a device sensor may be configured with a wicking surface or material that functions without a wicking coupler (such as an immobilized aptamer layer which is hydrophilic, or polymer ionophore layer which is porous to the analyte). A wicking coupler may be part of the same component or material that serves other purposes (e.g., a wicking collector or a wicking pump), and in such cases, the portion of said component or material that, at least in part, couples sweat to a sensor(s) and that is on or adjacent to the sensor(s), is also a wicking coupler as defined herein.

“Wicking space” refers to the space between the skin and wicking collector that would be filled by air, skin oil, or other non-sweat fluids or gases if no sweat existed. In some embodiments of the disclosed invention, even if sweat exists, the wicking collector removes some or most of sweat from the wicking space by action of wicking pressure provided by the wicking collector.

### DETAILED DESCRIPTION OF THE INVENTION

To clarify the proper numerical values or representations of sweat sampling rate and therefore chronological assurance, sweat generation rate and sweat volumes will be described in detail. From Dermatology: an illustrated color text, 5th ed., the maximum sweat generated per person per day is 10 L, which on average is 4 μL per gland maximum per day, or about 3 nL/min/gland. This is about 20× higher than the minimum sweat generation rate. The maximum stimulated sweat generation rate according to Buono 1992, J. Derm. Sci. 4, 33-37, “Cholinergic sensitivity of the eccrine sweat gland in trained and untrained men,” the maximum sweat generation rate by pilocarpine stimulation is about 4 nL/min/gland for untrained men and 8 nL/min/gland for trained (exercising often) men. Sweat stimulation data from “Pharmacologic responsiveness of isolated single eccrine sweat glands,” by K. Sato and F. Sato, Am. Physiological Society, Jul. 30, 1980, suggests a sweat generation rate up to about 5 nL/min/gland is possible with stimulation, and several types of sweat stimulating substances are disclosed (the data was for extracted and isolated monkey sweat glands, which are very similar to human ones). For simplicity, we can assume for calculations in the present disclosure (without so limiting the disclosure), that the minimum sweat generation rate is about 0.1 nL/min/gland, and the maximum sweat generation rate is about 5 nL/min/gland, which is about a 50× difference between the maximum and minimum rates.

Based on the assumption of a sweat gland density of 100/cm², a sensor that is 0.55 cm in radius (1.1 cm in diameter) would cover about 1 cm² area, or approximately 100 sweat glands. Next, assume a sweat volume under a skin-facing sensor (space between the sensor and the skin) of 100 μm average height or 100E-4 cm, and that same 1 cm² area, which provides a sweat volume of 100E-4 cm³ or about 100E-4 mL or 10 μL of volume. With the maximum sweat generation rate of 5 nL/min/gland and 100 glands, it would require a 20 minutes to fully refresh the sweat volume (using first principles/simplest calculation only). With the minimum sweat generation rate of 0.1 nL/min/gland and 100 glands, it would require 1000 minutes or ˜17 hours to refresh the sweat volume. Because the flow is not entirely centered, according to Sonner, et al., in Biomicrofluidics, 2015 May 15; 9(3):031301. doi: 10.1063/1.4921039, the time to fully refresh the sweat volume (i.e., new sweat replaces all old sweat) could be 6× longer or more. For slow sweat flow rates, back-diffusion of analytes and other confounding factors could make the effective sampling interval even larger. Clearly, conventional wearable sweat sensing approaches with large sweat volumes and slow sampling rates would find continuous sweat sample monitoring to be a significant challenge.

Sweat stimulation, or sweat activation, can be achieved by known methods. For example, sweat stimulation can be achieved by simple thermal stimulation, chemical heating pad, infrared light, by orally administering a drug, by intradermal injection of drugs such as carbachol, methylcholine or pilocarpine, and by dermal introduction of such drugs using iontophoresis, by sudo-motor-axon reflex sweating, or by other means. A device for iontophoresis may, for example, provide direct current and use large lead electrodes lined with porous material, where the positive pole is dampened with 2% pilocarpine hydrochloride or carbachol and the negative one with 0.9% NaCl solution. Sweat can also be controlled or created by asking the device wearer to conduct or increase activities or conditions that cause them to sweat.

The present disclosure applies at least to any type of sweat sensing device that stimulates sweat, measures sweat, sweat generation rate, sweat chronological assurance, its solutes, solutes that transfer into sweat from skin, a property of or things on the surface of skin, or properties or things beneath the skin. The disclosed invention, in all embodiments, includes at least one sensor that is specific to an analyte in sweat. To clarify further, just measuring sweat conductivity is not specific to one analyte because it measures the sum of conductance contributed by all ionic solutes in sweat. However, an ion-selective electrode configured to detect potassium is a sensor specific to one analyte. As an additional example, a sensor for sweat cortisol that only has interference (non-specificity) to estrogen, would still be specific to one analyte as described herein, since there are many device applications in which estrogen concentrations are static, but cortisol concentrations would change, making the sensor effectively specific to cortisol. Any suitable sensor may be used in the disclosed invention (e.g. ion-selective, enzymatic, antibody, aptamer, optical, electrical, mechanical, etc.). The disclosure applies to sweat sensing devices with various configurations including patches, bands, straps, portions of clothing, wearables, or any suitable mechanism that reliably brings sweat stimulating, sweat collecting, and/or sweat sensing technology into intimate proximity with sweat as it is generated. Some embodiments use adhesives to hold the device near the skin, but devices may also be secured by another suitable mechanism, such as a strap or helmet suspension.

Certain embodiments of the disclosure describe sensors as simple individual elements. It is understood that many sensors require two or more electrodes, reference electrodes, or additional supporting technology or features that are not captured in the description herein. Sensors are preferably electrical in nature, but may also include optical, chemical, mechanical, or other known biosensing mechanisms. Sensors can be in duplicate, triplicate, or more, to provide improved data and readings. Sensors may be referred to by what the sensor is sensing, for example: a sweat sensor; an impedance sensor; a sweat volume sensor; a sweat generation rate sensor; or a solute generation rate sensor. Certain embodiments of the disclosed invention show sub-components that may require additional obvious sub-components for use of the device in various applications (such as a battery), and for purpose of brevity and focus on inventive aspects are not explicitly shown in the diagrams or described in the embodiments of the present disclosure. As a further example, many embodiments of the disclosed invention may benefit from mechanical or other means to keep the devices or sub-components firmly affixed to skin or to provide pressure facilitating constant contact with skin or conformal contact with ridges or grooves in skin, as are known to those skilled in the art of wearable devices, patches, bandages, or other technologies or materials that are affixed to skin. Such means are included within the spirit of the disclosed invention. The present application has specification that builds upon PCT/US13/35092, the disclosure of which is hereby incorporated herein by reference in its entirety.

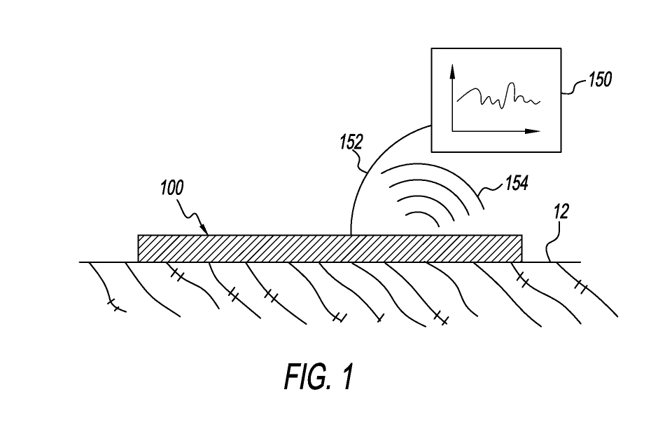

With reference to FIG. 1, a sweat sensor device 100 is placed on or near skin 12. In an alternate embodiment, the sweat sensor device may be simply fluidically connected to skin or regions near skin through microfluidics or other suitable techniques. Device 100 is in wired communication 152 or wireless communication 154 with a reader device 150. In some embodiments, reader device 150 may be a smart phone or portable electronic device. In alternate embodiments, device 100 and reader device 150 can be combined. In further alternate embodiments, communication 152 or 154 is not constant and could be a one-time data transmission from device 100 once it has completed its measurements of sweat.

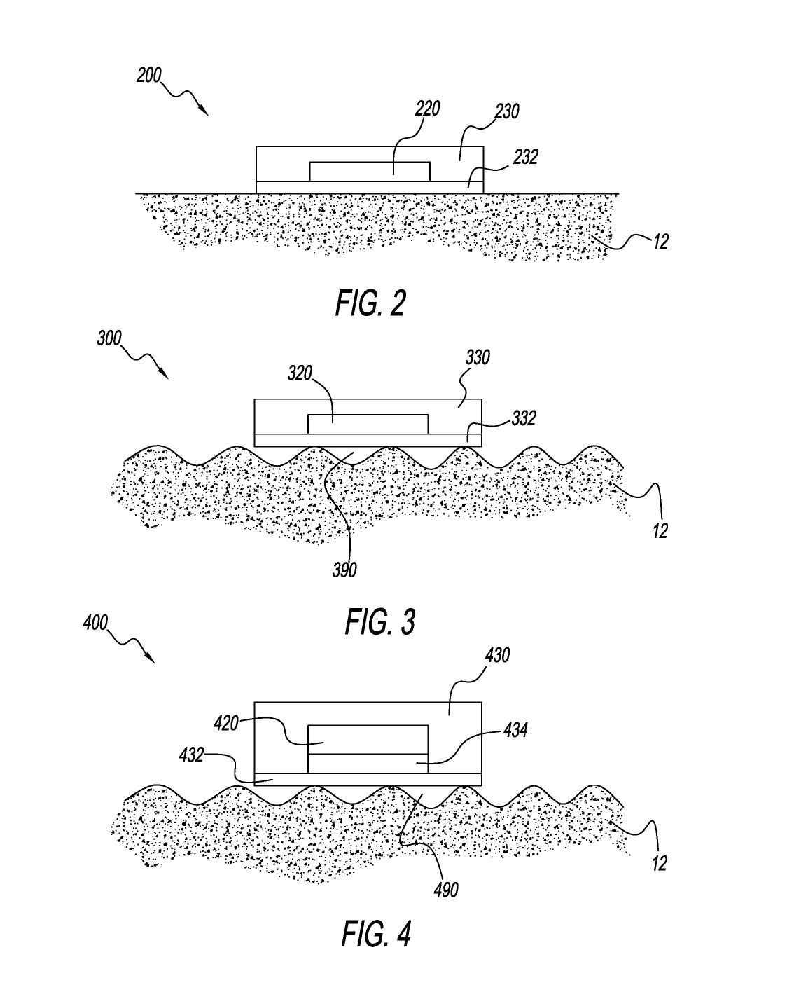

With reference to FIG. 2, device 200 includes at least one analyte-specific sweat sensor 220, at least one wicking collector 232, and at least one wicking pump 230. In order to ensure the sensor 220 remains wetted with sweat, some embodiments of the disclosed invention will be configured with a wicking collector 232 that has a wicking pressure equal to or greater than that of the wicking pump 230.

With reference to FIG. 3, device 300 includes at least one sensor 320, at least one wicking collector 332, at least one wicking pump 330, and a wicking space 390 between skin 12 and wicking collector 332. In order to reduce the sweat volume by creating at least a portion of a volume-reduced pathway, some embodiments will be configured with a wicking pump 330 that has greater wicking pressure than wicking space 390. In other embodiments, both wicking pump 330 and wicking collector 332 will have greater wicking pressure than wicking space 390. In a preferred embodiment, the disclosed invention reduces the sweat volume so that during operation of the sweat sensing device, wicking space 390 fills with less than 10% sweat (e.g., is 90% air or gas). Other embodiments may reduce sweat volume to a lesser extent, so that the wicking space 390 contains 20%, 30%, 40%, or up to 50% sweat (i.e. the sweat volume for the wicking space 390 is at least halved).

With reference to FIG. 4, device 400 includes at least one sensor 420, at least one wicking collector 432, at least one wicking coupler 434, at least one wicking pump 430, and a wicking space 490 between skin 12 and a wicking collector 432. In order to reduce the sweat volume by creating at least a portion of a volume-reduced pathway, and to ensure the sensor 420 remains wetted with sweat, some embodiments will be configured with a wicking coupler 434 that has a wicking pressure equal to or greater than that of the wicking pump 430. Otherwise, wicking pump 430 could remove an excess amount of sweat from sensor 420, preventing the sensor from performing accurate sweat measurements. However, for applications seeing higher sweat rates, the wicking pressure of wicking coupler 434 could be less than that of wicking pump 430, because enough sweat would be present to keep sensor 420 wetted. For configurations with a wicking coupler 434, the sweat sensing device will function regardless of the relative wicking pressures of wicking pump 430 and wicking collector 432. Similarly, with a wicking coupler 434, the disclosed device will function regardless of the relative wicking pressures of the wicking pump 430 and the wicking space 490.

The above-described configurations represent a basic foundation for either a simple device or a more complex device. Some embodiments of the disclosed invention may therefore include additional materials, components, designs, or other features for operation, as long as the device uses at least one wicking component, or operates at least in part by wicking pressure. More generally, regardless of how a wicking collector, a wicking pump, or a wicking coupler are configured, arranged, or omitted from a device of the present disclosure, the wicking pressure(s) are such that the sensor(s) is able to receive adequate sweat to perform accurate measurements during device operation.

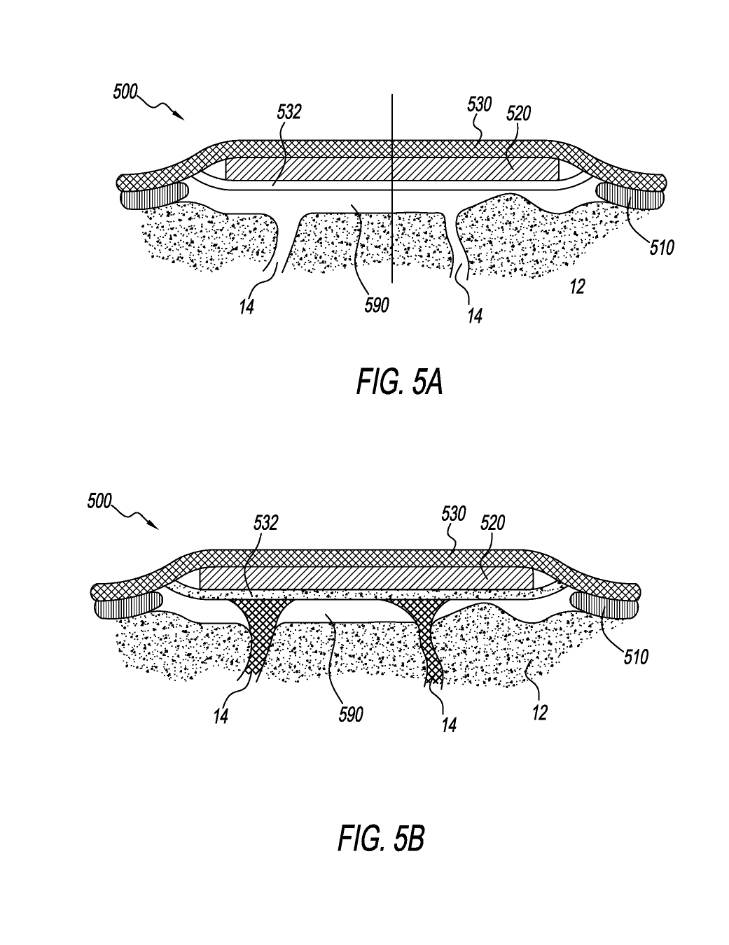

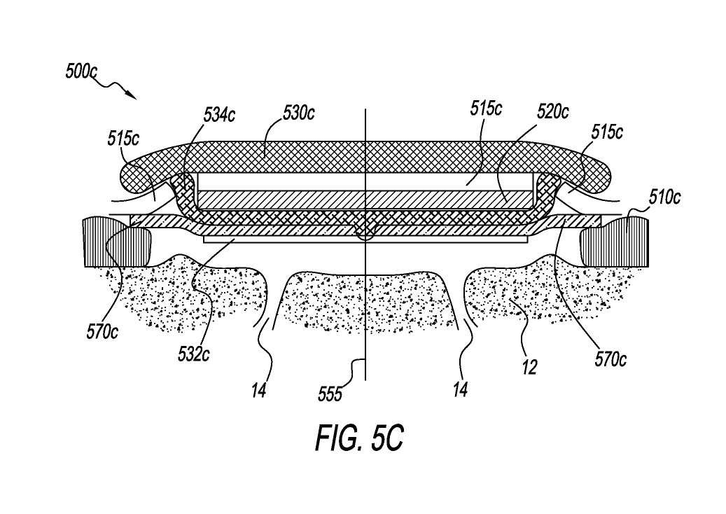

With reference to FIG. 5A, like numerals refer to like features of previous figures (e.g., 530 is a material like material 430 of FIG. 4). The device 500a includes a volume-reducing component comprised of a wicking collector 532. The wicking collector 532 could be any material that satisfies at least two criteria:

(1) Wicking collector 532 has a greater wicking pressure than wicking space 590. With reference to FIG. 5B, as sweat emerges onto skin 12 it will contact wicking collector 532 forming a sweat volume 580 which excludes a portion of the wicking space 590. Materials capable of providing adequate wicking pressure are well-known by those skilled in the art of materials science and microfluidics. Further, those skilled in the art can alter a material's wicking pressure to a desired level through control of capillary geometry, or through surface energy control.

(2) In the presence of sweat, the wicking collector 532 will tend to become saturated, at which point its wicking pressure would approach zero, and its ability to provide a reduced sweat volume would be compromised. Therefore, sweat must be continuously removed to prevent wicking collector 532 from becoming saturated. To remove excess sweat, the device 500a may be configured with a wicking pump 530 that is in fluid communication with wicking collector 532. In order to ensure adequate wicking pressure by wicking collector 532 and to maintain sufficient sweat sample contact with sensor 520, the wicking collector 532 will have wicking pressure greater than or equal to that of wicking pump 530. In addition, wicking pump 530 must have sufficient volume to sustain operation of the device throughout the application's intended duration (i.e., it must not become saturated during device operation). For example, if the device is to be used for 24 hours, then neither wicking collector 532 nor wicking pump 530 should become fully saturated with sweat during the 24 hours of operation. In some embodiments, wicking collector 532 and wicking pump 530 may be the same material or component.

With further reference to FIG. 5A, some embodiments of the disclosed invention may be configured with a wicking collector that meets a third optional criterion:

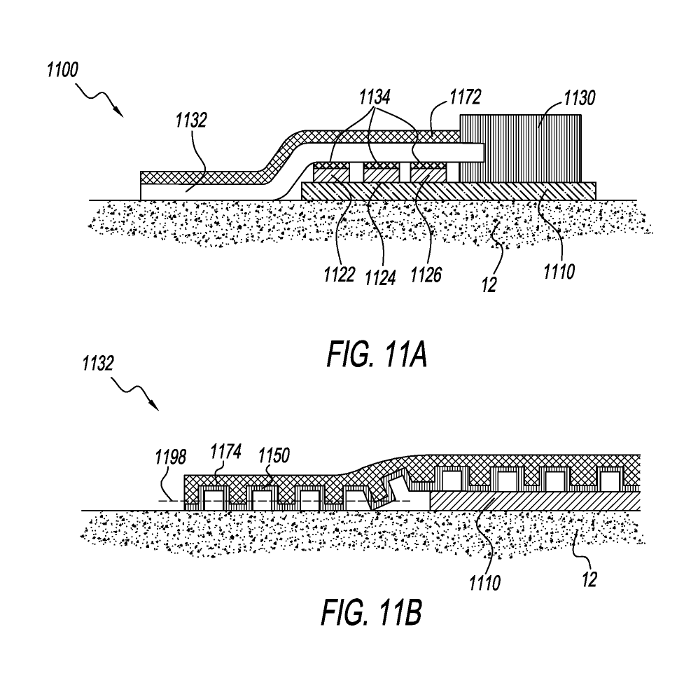

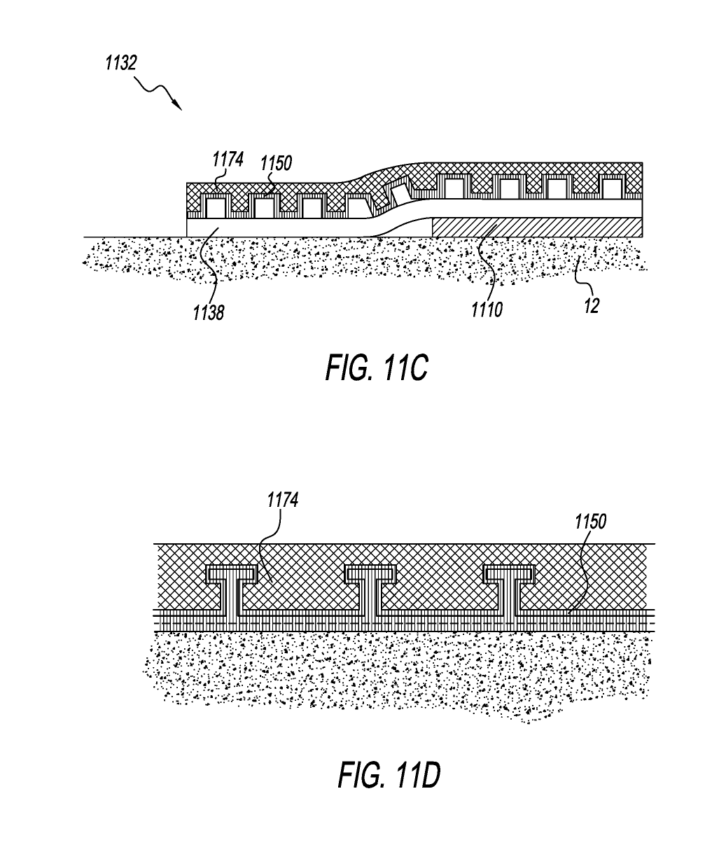

(3) The wicking collector 532 must be adequately thin, small in area, or non-porous so that its sweat volume is less than the sweat volume of the wicking space 590. Otherwise, adding a wicking collector 532 would primarily increase the sweat volume, which would tend to increase the chronologically assured sweat sampling interval. (There are instances where a low-volume wicking collector would not be critical, such as where the sweat sensor device is applied loosely to skin. However, in such cases the sweat volume would already be impractically large). Textiles, paper or other common wicking materials will generally fail this criteria, since they are typically more than 100 μm thick. A suitable implementation of the disclosed device would have, for example, a wicking space 590 with an average height of 50 μm due to skin roughness (more if hair or debris is present), and a wicking collector 532 comprised of a 5 μm thick layer of screen printed and hydrophilic nano-cellulose. In this example, the sweat volume would be reduced by roughly 10× over a similar device with no wicking collector. Other methods and materials to form a suitable wicking collector are also in the spirit of the present disclosure. Some embodiments of the present disclosure will require a further clarification of the optional volume relationship between wicking collector 532 and wicking space 590. As will be described in later embodiments and figures, e.g., FIG. 11, some embodiments will include a wicking collector 532 with at least a portion of its area that does not interface with, or is not adjacent to, skin. For such embodiments, only the portion of the wicking collector 532 that interfaces with or is adjacent to skin should have a sweat volume that is less than that of wicking space 590.

In other embodiments, a sweat sensing device may also include a wicking coupler. In one embodiment, the wicking coupler will have greater wicking pressure than the other wicking components. The relative pressures of the wicking collector 532 and wicking pump 530 to the wicking coupler can be immaterial to device operation, however, the pump must have greater wicking pressures than wicking space 590.

With reference to FIG. 5A, the volume of wicking space 590 can change over time due to skin plasticity, for example, skin can swell and become smoother as it hydrates, and skin can flatten if a sweat sensing device applies pressure against the skin surface. Therefore, for interpreting the disclosed invention, to reduce sweat volume between the skin 12 and the area of the wicking collector 532 that is on or adjacent to skin, at the time of first application of the device to skin, the sweat volume of the portion of the wicking collector 532 that interfaces with or is adjacent to skin, is less than the sweat volume of the space 590.

With further reference to FIG. 5A, saturating the wicking material with sweat can also be problematic, as it could increase the sweat volume of the wicking material. For example, the wicking material could be constructed of Rayon or other materials which have two or more levels of wicking pressures. For example, Rayon has a first and greater wicking pressure when fluid is wicked along grooves in its fibers, and a second and lower wicking pressure when fluid also fills the spaces in between such fibers. Alternately, an open-faced rectangular micro-channel could have a higher wicking pressure when it has less sweat in the channels (i.e., when only wicking along the corners of the channels—which have the highest wicking pressure—instead of filling the channels). Therefore, the invention may include a wicking element or material where the sweat volume during use is less than 50% of the total volume of such element or material, and which satisfies the other wicking pressure criteria and requirements as taught herein.

With reference to FIG. 5C, where like numerals refer to like features of previous figures, device 500c functions similarly to the embodiments illustrated in FIGS. 5A and 5B, but additionally is configured to provide a sweat flow that is centered on sensor 520c, referred to herein as sensor-centered sweat flow. The device 500c further includes sweat impermeable materials 515c and 570c, such as polymer films, where 515c may also serve as a substrate for fabrication of the device (e.g., one or more layers shown in FIG. 5C could be fabricated on the substrate, which may be made of Kapton or other suitable material). Component 570c has an opening in the center, marked by axis 555c, to allow sweat flow. For embodiments using circular sensors, having a sensor-centered sweat flow optimizes the chronologically assured sweat sampling rate for a given sweat generation rate, providing sampling rates as much as ˜6× faster than a non-centered flow, as taught by Sonner, et al. For embodiments using non-circular sensors, a centered sweat flow would serve a similar purpose.

Following the principles taught for FIG. 5A and FIG. 5B, wicking collector 532c is placed on the skin-facing surface of sweat impermeable film 570c. In a preferred embodiment of the invention, wicking coupler 534c would have a wicking pressure greater than or equal to that of the wicking pump 530c, so that sensor 520c remains wetted with sweat. In other embodiments, wicking coupler 534c would only require a wicking pressure greater than or equal to that of wicking pump 530c and wicking space 590c, and wicking collector 532c would only require greater wicking pressure than wicking space 590c. The sensor 520c could also be ring- or toroid-shaped, so that sweat can permeate through the sensor and eventually wick into wicking pump 530c. This configuration would require sweat impermeable material 515 to have an opening (not shown) centered on the sensor.

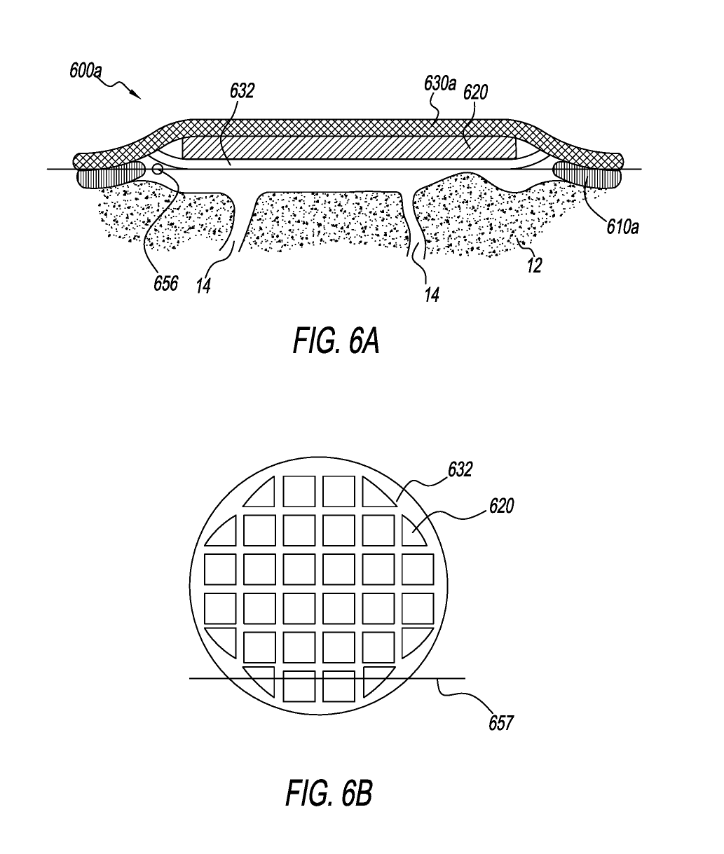

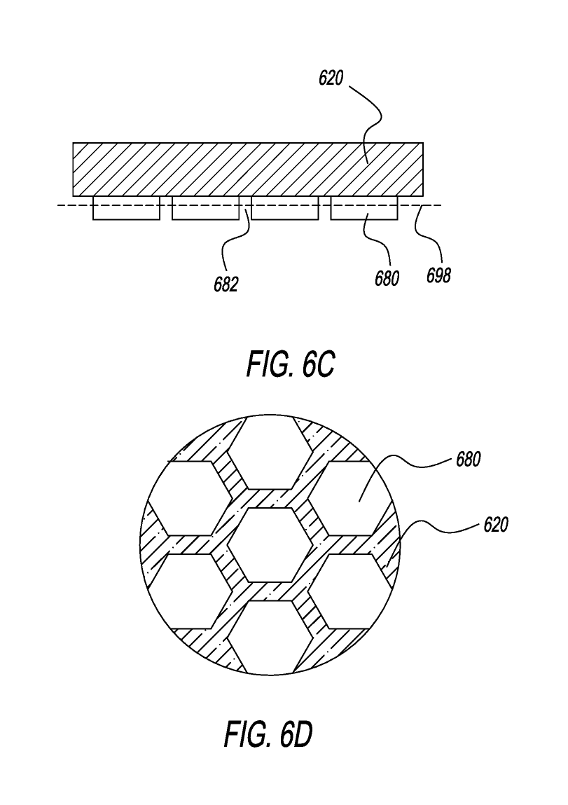

With reference to FIG. 6A, where like numerals refer to like features of previous figures, device 600a includes a wicking collector 632 configured as a network or grid of wicking pathways, and made from a textile, a hydrogel (e.g., agar), combinations thereof, or other material that further reduces sweat volume. Sensor 620 could be an ion-selective electrode that is able to properly function even if its surface is not fully wetted with sweat. FIG. 6B depicts a bottom view of device 600a from the perspective of planar reference 656. Wicking collector 632 has constraints as taught previously for FIG. 5, but has a reduced horizontal surface area compared to a continuous (non-grid) wicking material. Preferably, the grid portion of wicking collector 632 comprises <50% of the available horizontal surface area so that the effective sweat volume of the device is reduced by a factor of 2× compared to a continuous wicking material. In a preferred embodiment, the cross-sectional area of wicking material or channels in the wicking collector may comprise less than 50%, <30%, <20%, or <10% of the wicking collector's total cross-sectional surface area, or the the grid portion of wicking collector 632 comprises <50%, <30%, <20%, or <10% of the available surface area where it is on or adjacent to skin (see FIG. 6C and description for another example of cross-sectional area), which would reduce effective sweat volume by roughly 3×, 5×, or 10×, respectively. While greatly reducing effective sweat volume, including open areas in wicking collector 632 could have the undesirable effect of reducing the signal measured by sensor 620, since the sensor's sweat contact area is determined by the area of contact with wicking collector 632. Therefore, in an alternate embodiment, wicking collector 632 could be used in place of wicking collector 532c in FIG. 5C. Using sensor-centered sweat flow, the open areas of wicking collector 632 would not directly effect the sensor's sweat contact area.

With reference to FIG. 6C and FIG. 6D, an alternate wicking collector embodiment is shown by a view along cross-sectional axis 657. In this embodiment, sweat wicking is done by a partial microfluidic channel (such as a rectangular micro-channel), depicted here as a channel 682, which is part of a network of wicking channels which are at least partially open to the skin surface, such as the hexagonal network shown in FIG. 6D. These channels may also have reduced cross-sectional area, along the dimension marked by dotted lines 698 (again, the cross-sectional area of wicking material or channels in the wicking collector comprise less than 50% of the wicking collector's total cross-sectional surface area). The surfaces of the channel 682 may be hydrophilic, so that sweat rapidly wicks into and along the channel at least in part by pressure driven flow similar to capillary flow. For example, material 680 could be a simple hydrophilic polymer, or a polymer like PET that is treated or coated to be hydrophilic or super-hydrophilic, e.g., with a coating of a nano-silica or a hydrogel, such as agar. The intersections between channels promote continuous wetting from channel to channel (overcoming pinning forces, for example). In some embodiments, the channels may be at least 50% enclosed, with openings to allow sweat to enter the microfluidic channels, as will be taught in greater detail for FIG. 12C.

With reference to FIGS. 6B to 6D, the wicking network or channels as disclosed offer the advantage of redundancy over other possible configurations. For example, a wicking collector could use continuous channels, such as a spiral-shaped single channel design. However, any defect in such continuous channel configurations could disrupt the wicking and sweat transport capability of the entire wicking collector. However, the wicking networks described herein provide redundancy in potential wicking paths, meaning that a broken sub-channel will not prevent the network from wicking and transporting sweat. Therefore, embodiments of the disclosed invention may include a network of at least partially redundant wicking pathways.

With reference to FIGS. 6B to 6D, the wicking network as disclosed offer the advantage of greater contact area with the openings of sweat glands on the skin surface. A simple textile with random fiber arrangement (e.g. non-woven) could have areas with poor local contact to skin, and therefore in some areas would require more sweat volume in order to allow wicking connection between the opening of a sweat gland on the skin surface and the textile. The wicking network of FIG. 6B can be precisely configured such that they have no more than 500 μm and preferably no more than 100 μm distance between adjacent wicking pathways in the wicking network of FIG. 6B.

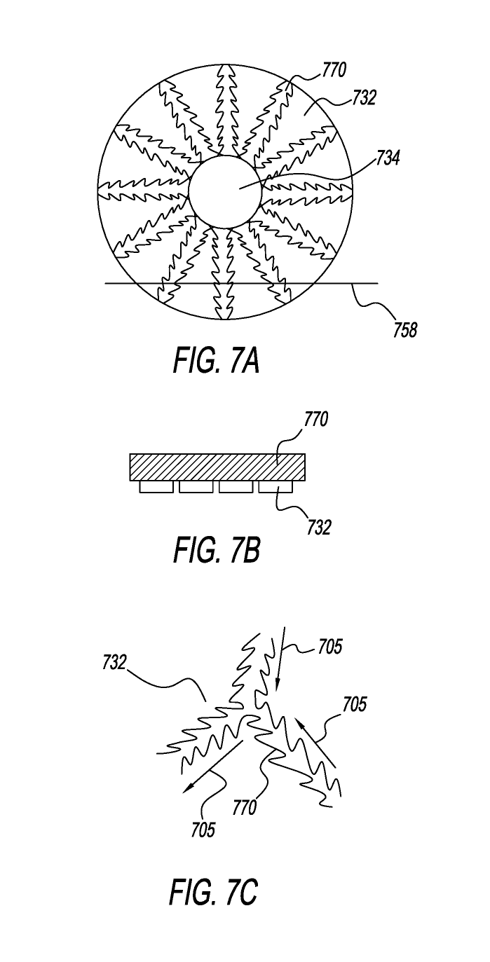

FIGS. 7A and 7B depict an alternate embodiment that may optimally be applied to devices that use sensor-centered sweat flow, such as is depicted in FIG. 5C, but also illustrates principles which may be applied more generally to embodiments of the disclosed invention. FIG. 7A depicts a top-view diagram of wicking collector 532c from FIG. 5C, while FIG. 7B is a horizontal view along cross-section 758 in FIG. 7A. With reference to FIG. 7A, where like numerals depict corresponding features of previous figures, rather than a solid or open area design, wicking collector 732 is a hydrophilic material, such as a polymer with at least one defined trench to facilitate sweat flow, where the bottom of the trench(es) is the surface of the sweat impermeable film 770. A central cut-out is also depicted, illustrating a connection to wicking coupler 734, which is in fluid communication with the trench(es). A primary advantage of this embodiment is that the trenches can be designed with a geometry that promotes unidirectional capillary flow. With unidirectional flow, when sweat wets wicking collector 732 at a point between the outer radius and the wicking coupler 734, the sweat would wick to the radial center and the remainder of the trench would not wet with sweat. This embodiment, therefore, reduces the sweat volume of wicking collector 732 relative to a continuous wicking material, and offers further reduced sweat volume since typically only a portion of the trenches (if configured with more than one) will fill with sweat during device use.

In other embodiments, unidirectional capillary trenches may be configured in a “tree root” or other suitable pattern to provide more efficient sweat transport in the area covered by wicking collector 732, or to optimize trench coverage of sweat ducts under the wicking collector, assuming a random pattern of sweat duct placement. For example, FIG. 7C depicts a close-up view of a tree root trench pattern, with fluid flow direction noted by arrows 705. In such a configuration, trenches 770 have width and depth of roughly 5 μm, and comprise approximately 10% of the total area of wicking collector 732. Due to sweat gland location, only perhaps 10% of the trench total area would be wetted by sweat, so that compared to a 50 μm wicking space 590, the effective sweat volume would be reduced by 1000×. Other unidirectional microfluidic flow or wicking components providing similar advantages may also be applied, as are known by those skilled in the art of microfluidics.

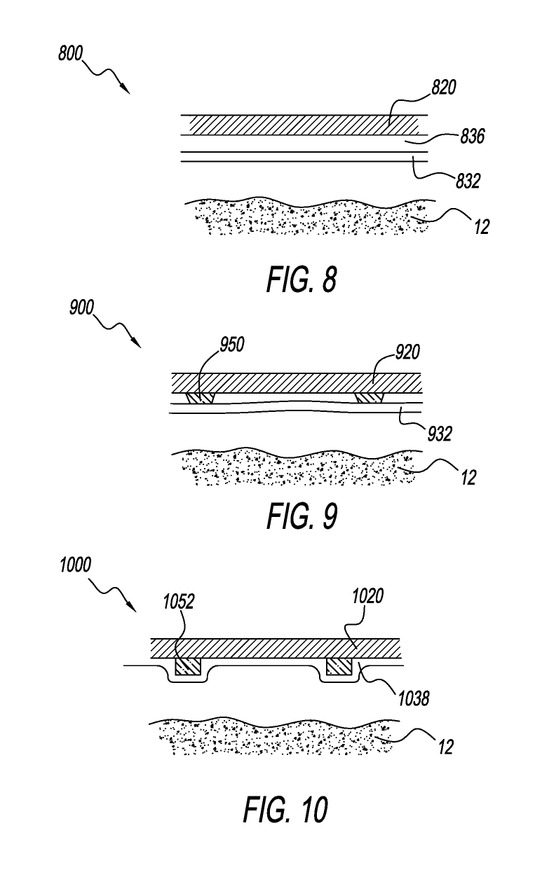

With reference to FIG. 8, in the disclosed invention, wicking components such as those depicted in FIGS. 5A and 5B may also serve as sensor protection components. Wicking sensor protection materials must wick sweat and protect a sensor from damage caused by placement on skin, such as abrasion, puncture, oil exposure, sensor fouling, or other forms of potential damage to the sensor. A portion of a device 800 includes a material, such as a hydrogel, that protects sensor 820 from abrasion by skin 12 or from abrasion by wicking collector 832 (e.g., 832 could be a thin sheet of nanocellulose or non-woven polymer porous material). During use of the sweat sensor device, skin 12 or wicking collector 832 could move horizontally and abrade against sensor 820. However, with the inclusion of protective material 836, the protective material would shield the sensor surface from abrasion or other damage. A variety of materials may be used for component 836 or 832, as long as the material is capable of adhesion to the surface of the sensor and is porous enough to allow the sensor to function. Non-limiting examples include an aerogel, a low density gel, dialysis membrane material, a porous polymer, nafion, or an in-situ deposited or electro-deposited polymer that is porous and deposited onto the sensor. For example, component 836 could be a strongly wicking hydrogel adhered to sensor such as agar gel, and if component 836 is is fragile then component 832 could be a non-woven nylon micro-mesh which protects component 836 from abrasion.

With reference to FIG. 9, in another disclosed embodiment, a partial view of sweat sensing device 900 includes a wicking collector 932 that wicks sweat and is fixed by adhesive or mounting 950 to sensor 920. In this embodiment, abrasion by horizontal movement of material 932 is mitigated, and the open areas outside of mounting 950 are sufficient to allow sweat to wet sensor 920 so that sensing can occur. Because wicking collector 932 is not in intimate contact with sensor 920, both components may require treatment to ensure the sensor wets with sweat, for example, both the wicking collector and sensor may be vacuum dipped and coated with a thin layer of water-soluble polymer, such as poly-ethylene oxide, or they may be treated with another coating that is sweat soluble and promotes wicking, or they could be treated with a non-dissolvable polymer, like agar gel.

With reference to FIG. 10, in another disclosed embodiment, a partial view of sweat sensing device 1000 depicts a spacer 1052 mounted on sensor 1020 that holds most of the sensor's surface away from abrasive contact with skin 12 or other material (not shown). If sweat is not adequately wicking to the sweat sensor surface, a wicking collector 1032 may be included to wick sweat to sensor 1020. Spacer 1052 may be a plurality of posts, a grid, or other components that prevent abrasive contact between the sensor and skin 12 or other abrading material, while also allowing sweat to wet against sensor 1020. Another advantage of the spacer 1052 is that the wicking collector 1032 will have less contact with the skin surface, and therefore will receive less contamination from the skin's surface, resulting in higher quality sensor data. Therefore, the disclosed invention may include at least one spacer component that separates the majority of a wicking collector's surface area from skin contact.

With reference to FIG. 11A, a device 1100 includes a wicking collector 1132, an evaporation prevention cover 1172, at least one sensor(s) 1122, 1124, 1126, a substrate 1110, a wicking coupler 1134, and a wicking pump 1130. Some wicking materials used in a wicking collector 1132 may allow sweat collection at flow rates that are extremely low, and therefore significant sample evaporation could occur before sweat reaches the sensor(s) 1122, 1124, 1126. To prevent unwanted evaporation, some embodiments may include a cover 1172 made of PET, other polymer, or other suitable material to at least partially block sweat from evaporating from wicking collector 1132. Some embodiments with a more rigid wicking collector 1132 (e.g., micro-channels embossed in a thick film of PET), or embodiments using sensor(s) 1122, 1124, 1126 with surfaces that are sensitive to abrasion, may require a wicking coupler 1134 between the wicking collector and sensor(s). In such embodiments, wicking coupler 1134 is any material included between sensor(s) 1122, 1124, 1126 and wicking collector 1132 that promotes wetting the sensor(s) with sweat, or that protects the sensor(s) from abrasion. For example, a wicking coupler 1134 made of hot-coated agar-gel can be applied to the sensor(s) at a thickness in the range of 1's to 100's of μm. A further advantage of using a wicking coupler 1134 as disclosed is that the sensor(s) may deviate from a highly planar surface geometry and still remain in fluidic contact with wicking collector 1132.

With further reference to FIG. 11A, as discussed above, the various wicking components of device 1100 must be configured with proper relative wicking pressures to allow proper device function. For example, wicking coupler 1134 should have a wicking pressure that is greater than or equal to that of wicking collector 1132, or wicking pump 1130. This requirement is to ensure that the sensor(s) 1122, 1124, 1126 always remain wetted with sweat during device operation. In other embodiments, the sensor(s) themselves may not require a wicking coupler if they have a wicking surface or material that functions as a wicking coupler (e.g., an immobilized hydrophilic aptamer layer on a rough or textured electrode surface, or polymer ionophore layer which is porous to the analyte even if not fully contacted by sweat at its entire surface area).

With reference to FIG. 11B, some embodiments of the disclosed invention may include a wicking collector 1132 comprised of a network of hydrophilic channels created, for example, by hot-embossing channels into the surface of a polymer (e.g., PET) component 1174 and coating the channels with hydrophilic gold 1150. The network of channels can take on features, shapes, or functions previously described, e.g., in FIGS. 6B, 6D, 7A, and 7C. As configured, the hydrophilic gold coating 1150 promotes wicking of sweat to the device sensors, but may also function as an impedance electrode capable of measuring skin impedance. Skin impedance, in turn, provides an indirect assessment of changes in sweat generation rate and sweat salinity. Therefore, the disclosed invention may include a wicking collector that further includes at least one electrode. In other embodiments, coating 1150 may be at least partially composed of a sensor coating, such as an ionophore, immunoassay, aptamer, or other coating, so that coating 1150 could detect a sweat analyte. Having sensor capability on the wicking collector may be useful for device applications in which a sweat analyte needs to be measured as it emerges from skin, for example, a protein that degrades quickly in sweat (due to enzymes, oxidation, etc.). Therefore, the wicking collector 1132 may include at least one sensor specific to at least one sweat analyte.

With reference to FIG. 11C and FIG. 11D, some embodiments may include additional components that improve sweat movement along wicking collector channels. For example, at junctions between multiple channels, diverging capillary geometries and pinning forces can hinder or stop fluid flow. Accordingly, in an alternate embodiment, the channels may be capped with an additional material that is also sweat porous, such as track-etch membrane 1138 shown in FIG. 11C, or using an alternate channel geometry as shown in FIG. 11D. With reference to FIG. 11C, typically, more than 90-95% of the track-etch membrane 1138 is flat, which improves the ability for sweat to wick horizontally past junctions or possible pinning features. Because track etch membranes are porous vertically, sweat can still enter the network of channels through membrane 1138. As disclosed, the invention may therefore include a network of wicking channels that are primarily closed to the skin surface by being at least partially covered, meaning at least 50% of their potential skin-facing surface will face solid portions of membrane 1138.

The following examples are provided to help illustrate the disclosed invention, and are not comprehensive or limiting in any manner.

### EXAMPLE 1

This first example provides a hypothetical calculation of wicking pressures for elements of the disclosed invention. For purposes of the calculation, the wicking coupler will have the greatest wicking pressure, followed by the wicking pump, and lastly the wicking collector. These relative wicking pressure strengths will ensure that sweat is continuously removed from the wicking collector so that negligible sweat remains on the skin surface.

In all our calculations, our wicking pressures all originate from negative Laplace pressure Δp=γ(1/R₁+1/R₂), where sweat's surface tension is close to that of pure water: γ˜70 mN/m. For simplicity, assume the fluid is constant, and principal radii, R₁ and R₂, are concave (negative). To further simplify the discussion, we will calculate effective radii for each sub-component (no need to quantify wicking pressure, smaller radii=larger wicking pressure).

The space between skin and a wicking collector. First is a rough 2D calculation of what is required to reduce the effective sweat volume to at least 10% of the available sweat volume. (Which will also reduce skin surface contamination). Assume 60 μm peak-to-valley skin roughness (which would be greater with hair or skin defects). If the wicking collector is touching the skin, to reduce the effective sweat volume to 10% of available volume, the sweat will be wicked into a space that extends only 20 μm out from the skin ridges (assuming triangular ridge shapes), with a meniscus between skin and the collector that spans ˜20 μm. Next, assume the most difficult scenario of a sweat contact angle on skin of θₛₖᵢₙ=0°, which represents a hydrated and swollen skin surface (more typical contact angles are around) 90°). Using a wicking collector made of polyamide (nylon, PA46) that is hot-embossed with a network of rectangular channels similar to those shown in FIGS. 6, 7, and 10, with an averaged contact angle of θ_{poly}=45°. The wicking pressure for a long triangular groove in skin will be dominated by a single radius of curvature (Rₛₖᵢₙ), which can be visualized along the meniscus edge, and calculated as Rₛₖᵢₙ=−h/(cos [θ_{poly}−45°]+cos θₛₖᵢₙ)≅10 μm where h is ˜20 μm as previously discussed and the “−45” term denotes a converging capillary feature or channel. Having calculated Rₛₖᵢₙ at ˜10 μm, the wicking pressure required from the wicking collector can be determined.

The wicking collector. Assume the wicking collector has square cross-section microchannels with a 1:1 aspect ratio and width w, for which an effective single capillary radius, R_{collector}, can be calculated as R_{collector}=w/(3 cos(θ_{poly})−1). The wicking pressure of the collector must sustain the <10% sweat volume between skin and the wicking collector, and therefore R_{collector}=Rₛₖᵢₙ=10 μm. This R_{collector} value yields a calculated channel width of ˜11-12 μm. A suitable material for the wicking collector is polyamide (nylon), because it is easily microreplicatable, hydrophilic, and relative to many other polymers, exhibits lower non-specific sweat protein and analyte binding. The wicking collector will initially be coated with a layer of poly-vinyl-alcohol (PVA) water-dissolvable polymer of 10's of nm thickness, to enable wetting past channel junctions.

The wicking coupler. Next, assume a 10's of μm thick wicking coupler between the wicking collector and a sensor. For device operation, the wicking coupler must keep the sensors continually wetted with a new sample of sweat. To achieve this will require at least a 10× decrease in effective capillary radius or R_{coupler}˜1 μm (this includes a margin of error to allow for possible variances). There are several materials available from which a wicking material with micrometer-scale capillaries may be fabricated, for example, a hydrophilized nano-cellulose material that is 20 μm thick when hydrated. Nano-cellulose forms a gel-like material which remains cohesive even when hydrated due to microfibril interactions. Nano-cellulose is soft and should promote wetting to sensors. Another attractive possibility is to coat and polymerize a thin film of a hydrogel or super-porous hydrogel. Hydrated hydrogels can have pore sizes sufficient to allow advective transport of even large proteins. Super-porous hydrogels have a physically open porous network that can be tuned from sizes of 100's of nm to several μm's. A hydrogel wicking coupler has further advantages because hydrogels (1) are pliant when wet and with slight pressure will remain in wetted contact with sensors; and (2) can be coated onto, and in some cases adhered to, the polyamide wicking collector or sensors.

The wicking pump. In this example, the pump serves primarily as a means to collect and dispose of excess sweat throughout device operation. The wicking pump must have greater wicking pressure than the wicking collector, but its wicking pressure must not exceed that of the wicking coupler or the pump will remove sweat from the wicking coupler and leave inadequate sweat on the sensors for accurate measurements. The wicking pump may be fabricated by simple techniques, such as stacking of a plurality of hydrophilic membrane filters such as those made of nitrocellulose or other membrane materials, and which have well-tuned pore sizes and wicking pressure; fairly homogeneous beads (e.g., commercial monodisperse Reade Silica powder); a longer-chain length hydrogel; micro/nano-porous sponges; or other suitable techniques that provide an effective wicking radius of Rₚᵤₘₚ=2-3 μm. Again, we could easily decrease the effective R_{coupler} to 10's or 100's of nM to allow a wider selection of materials and effective radius Rₚᵤₘₚ for the wicking pump. The pump can easily be designed to store 10's to 100's of μL of sweat, allowing for continuous use for >24 hours at 0.5 nL/min/gland. Note, the 10% volume between skin and the wicking collector could be further reduced by the wicking pressure of the wicking pump.

### EXAMPLE 2

The following example is taught for a wicking collector area of 0.1 cm², an active sweat gland density of 100 glands/cm² (which translates to 10 glands facing the wicking collector), and a sweat generation rate of 0.5 nL/min/gland (which translates to a total sweat flow rate to the collector of 50 nL/min/cm²). This example may be adapted to illustrate other gland densities, sweat generation rates, wicking collector areas, skin roughness, or alternate use scenarios or device designs.

Consider the device 500 of FIG. 5, with a hydrogel film wicking collector 5, 15, or 45 μm thick, and a hydrogel wicking pump of similar or lesser wicking pressure than the collector, and with a pumping capacity or volume of 100 μL. The volume of the wicking collector adjacent to skin is therefore 50, 150, or 450 nL, with sweat sampling intervals of 10, 30, and 90 minutes, respectively.

### EXAMPLE 3

Consider Example 2 above, but with the hydrogel containing 50% by volume of microbeads. The resulting sampling intervals are reduced to 5, 15, and 45 minutes.

### EXAMPLE 4

Consider Example 2 above, but with the hydrogel replaced by a grid of agar similar to that taught for FIG. 6. Assuming 10% of the sensor area is agar and 90% is open area, the resulting sweat sampling intervals are 1, 3, and 9 minutes.

### EXAMPLE 5

Consider Example 2 above, but with a sensor-centered sweat flow device 500c, as described in FIG. 5. Assume the wicking coupler 534c and wicking collector 532c are composed of a hydrogel and have the same thickness. The sweat sampling interval will again be of 10, 30, and 90 minutes, but will not require correction to account for non-centered sweat flow, as taught by Sonner, et al. There will also be as much as a 2× delay for when new sweat reaches the sensor, because twice as much wicking material now exists between the sensor and the skin surface.

### EXAMPLE 6

This example illustrates how to calculate and interpret the sweat sampling interval based on advective sweat flow. Consider a device 1100 similar to that described for FIG. 11, and with basic requirements similar to those taught for Example 2 (wicking collector area, glands, sweat generation rate, etc.). Assume rectangular microchannels that are 10 μm wide and 10 μm deep, arranged in a hexagonal network, and coated with a 100 nm hydrogel layer making the channels super-hydrophilic. If the network of channels comprises 10% of the area of the collector, the design will be equivalent to a continuous film wicking collector only 1 μm thick, with a volume of 10 nL. As noted previously, the wicking collector area on skin is 0.1 cm². Assume the portion of the wicking collector not on skin is 5 mm long and 200 μm wide (or an area of 0.01 cm²), and therefore has a volume of 1 nL. Assume each of at least one sensors is 200×200 μm in area, or 0.0004 cm², and each has a wicking coupler of agar gel that is 25 μm thick, so that the volume of the wicking coupler on each sensor is 1 nL. The sampling interval will be dominated by the volume of the wicking collector, and therefore the sweat sampling interval would be approximately 2 minutes. While beyond the scope of this example, large analytes, like proteins, may require additional time to diffuse through the wicking coupler, causing the sensor response times to be slower than two minutes.

This Example 5 can also represent an embodiment of the present disclosure where the space between skin and the wicking collector could have a very low volume, and the primary purpose of the disclosed invention is simply to reduce the volume of the wicking collector and to ensure that the sensor(s) remain wetted with sweat.

### EXAMPLE 7

Consider a device similar to the device in Example 5, but with an effective space between skin and the wicking collector of 50 μm in height. If the the requirement of the disclosed invention were not satisfied, specifically the wicking collector, wicking pump, and wicking coupler having greater wicking pressure than the wicking space, then the wicking space would be filled with sweat. The approximate time to refresh this volume with new sweat can be translated into sweat sampling interval, and using first order calculations of simply refilling that volume, the sampling interval would be 100 minutes. Therefore, the wicking principles disclosed herein can improve the sweat sampling interval by 50×. If a wicking collector or other elements, like a wicking pump, are added to reduce or eliminate the sweat volume associated with the effective 50 μm of space between skin and the collector, then the wicking collector should have an effective sweat volume of less than 50 μm in the area that it is on or adjacent to skin. Otherwise adding the wicking collector increases the total sweat volume, meaning its does not help reduce the sweat volume between the device and skin. This was stated previously for FIG. 5 as “The wicking collector 532 must be adequately thin, small in area, or non-porous so that its sweat volume is less than the sweat volume of the wicking space 590”, and can be more generally stated as: “the wicking collector has a sweat volume, in the portion of its area on or adjacent to skin, that is less than the sweat volume between the wicking collector and skin.”

### EXAMPLE 8

Consider again Example 2, but with several pumping capacities for the wicking pump. The wicking pump can be made from various materials (gels, textiles, membranes, beads, etc.) to meet the requirements as disclosed herein. With a total sweat flow rate to the collector of 5 nL/min, pumping durations of at least 3 hours, 6 hours, 12 hours, and 24 hours would be possible with capacities of at least 0.9 μL, 1.8 μL, 3.6 μL, and 7.2 μL, respectively. These volumes are all far less than one mL (1 cm³). Wicking pump capacities can be adapted to other gland densities, sweat generation rates, wicking collector areas, skin roughness, or alternate use scenarios or device designs. For example, if the sweat generation rate were doubled to 1 nL/min/gland, then wicking pump capacities listed above would need to be doubled as well.

This has been a description of the present invention along with a preferred method of practicing the present invention, however the invention itself should only be defined by the appended claims.

1. A sweat sensing device providing a reduced wicking volume, the sweat sensing device comprising:
a wicking space;
one or more volume-reducing components, wherein each volume-reducing component of the one or more volume-reducing components includes one or more of the following: a wicking collector, a wicking pump, and a wicking coupler; and
one or more sensors for measuring a characteristic of an analyte in sweat.

2. The device of claim 1, wherein a sweat flow pathway has a volume that is less than a volume of the wicking space, and at least one of the one or more volume-reducing components has a wicking pressure that is greater than a wicking pressure of the wicking space.

3. The device of claim 1, wherein at least a portion of an area of the wicking collector is on or adjacent to skin, and the area of the wicking collector has a volume that is less than a volume of the wicking space.

4. The device of claim 1, wherein the wicking space contains no more than 50% sweat by volume.

5. The device of claim 1, wherein:
the wicking collector has a wicking pressure that is greater than or equal to that of the wicking pump;
the wicking coupler has a wicking pressure that is greater than or equal to that of the wicking pump;
the wicking has a wicking pressure that is greater than or equal to that of the wicking space; or
the wicking collector has a wicking pressure that is greater than that of the wicking space.

6. The device of claim 1, wherein the wicking coupler has a greater wicking pressure than a wicking pressure of the wicking pump, the wicking pump has greater wicking pressure than a wicking pressure of the wicking collector, and the wicking collector has greater wicking pressure than a wicking pressure of the wicking space.

7. The device of claim 1, wherein a volume of sweat as a percentage of a total volume of at least one of the wicking collector, the wicking pump, and the wicking coupler is less than 50%.

8. The device of claim 1, wherein the one or more sensors receive a centered flow of sweat.

9. The device of claim 2, wherein the wicking collector has a cross-sectional surface area that includes a first portion comprised of a wicking component, and a second portion comprised of an open space, wherein a cross-sectional surface area of the first portion comprises less than 50% of a cross-sectional surface area of the wicking collector.

10. The device of claim 2, wherein the wicking collector includes a network of wicking channels, and wherein the network of wicking channels are at least partially open to a skin surface.

11. The device of claim 2, wherein the wicking collector includes a network of wicking channels, and wherein the network of wicking channels are at least partially closed to a skin surface.

12. The device of claim 2, wherein the wicking collector includes a network of wicking channels, and wherein the network of wicking channels includes a plurality of redundant channels.

13. The device of claim 2, wherein a distance between adjacent wicking pathways is less than 500 μm.

14. The device of claim 1, wherein the wicking collector includes one or more unidirectional wicking channels.

15. The device of claim 1, wherein the one or more sensors include one or more of: a sensor protection material and a spacer material.

16. The device of claim 1, wherein the device includes one or more of the following: one or more sensor protection components; one or more spacer components; one or more evaporation prevention components

17. The device of claim 1, wherein the wicking collector includes at least one electrode.

18. The device of claim 1, wherein the device, given that the wicking collector receives an average sweat flow rate of ˜50 nL/min/cm², is capable of one of the following chronologically assured sweat sampling intervals: <90 minutes; <45 minutes; <30 minutes; <15 minutes; <9 minutes; <5 minutes; <3 minutes; <2 minutes; <1 minute.

19-55. (canceled)
